(19) United States
(12) Patent Application Publication
(10) Pub. No.: US 2025/0319135 A1
(43) Pub. Date: Oct. 16, 2025

(54) METHOD FOR PREPARING EXOSOMES DERIVED FROM ANTLER PERIOSTEAL MESENCHYMAL STEM CELLS AND APPLICATIONS THEREOF

(71) Applicant: SHINE-ON BIOMEDICAL CO., LTD., TAICHUNG (TW)

(72) Inventors: YI-HUI LEE, TAICHNUG (TW); YU-CHENG LEE, TAICHUNG (TW); CHIEN-LIN CHEN, TAICHUNG (TW); HUNG-CHE CHIANG, TAICHUNG (TW); CHI-HAO CHANG, TAICHUNG (TW)

(21) Appl. No.: 19/077,210

(22) Filed: Mar. 12, 2025

(30) Foreign Application Priority Data
Apr. 16, 2024 (TW) 113114216

(51) Int. Cl. A61K 35/28; C12N 5/0775

(52) U.S. Cl. CPC A61K 35/28; C12N 5/0663; C12N 2509/00

(57) ABSTRACT

The present invention provides a method for preparing exosomes derived from antler periosteal mesenchymal stem cells and their applications. After obtaining antler periosteal tissue, the tissue is placed in a DPBS culture dish containing antibiotics for further processing. The periosteal tissue is then minced and washed with DPBS until it is free of blood. The minced tissue is treated with digestive enzymes and incubated in a carbon dioxide (CO₂) incubator to facilitate enzymatic digestion. Subsequently, alpha-MEM complete culture medium is added to further process the tissue. The digested tissue is filtered and centrifuged to isolate the primary periosteal cells, which are then counted and cultured in a suitable medium to obtain mesenchymal stem cells (MSCs) derived from antler periosteum. The cultured periosteal MSCs are further processed to isolate exosomes, which can be applied to promote the proliferation and repair of human fibroblasts, enhance skin quality, and stimulate hair growth.

### BACKGROUND OF INVENTION

### 1. Field of the Invention

The present invention relates to antler stem cells, and more specifically, it relates to a method for preparing exosomes derived from antler periosteal mesenchymal stem cells and their applications.

### 2. Description of Related Art

Mesenchymal stem cells (MSCs) are adult stem cells present in various tissues and organs, possessing multipotent differentiation potential. Due to their excellent immunomodulatory properties and low tumorigenicity, MSCs are one of the most frequently utilized cell types in stem cell applications.

Additionally, exosomes are vesicular structures secreted by stem cells during physiological processes. They contain complex RNA, proteins, and other bioactive molecules and play a crucial role in intercellular communication. Exosomes can be easily collected, stored, and transported with controlled quality, and in animal experiments, they exhibit therapeutic effects similar to those of mesenchymal stem cells. However, the composition of exosomes secreted by cells is not fixed; rather, it varies depending on cellular conditions and other influencing factors. Thus, identifying a cell source capable of producing exosomes in large quantities and with high stability is critical for achieving a major breakthrough in the application of exosome-based therapies.

Deer antlers have been used in traditional Chinese medicine (TCM) for centuries, and are known for their effects in tonifying essence and blood, strengthening muscles and bones, and enhancing vitality. Deer antler extracts have also been utilized in the treatment of various diseases. Compared with bone marrow mesenchymal stem cells and umbilical cord mesenchymal stem cells, which are commonly used in the field of biotechnology, antler stem cells (ASCs) are easier to obtain. These antler stem cells are mesenchymal stem cells derived from the antler periosteum, sharing the typical characteristics of MSCs.

Research indicates that antler stem cells exhibit greater proliferative capacity under in vitro culture conditions. While conventional mesenchymal stem cells typically sustain proliferation for about 15 passages, antler stem cells can be passaged up to 55 times while maintaining stable proliferative ability. Consequently, antler stem cells serve as a reliable and sustainable source for therapeutic exosomes.

Currently, exosomes derived from antler stem cells are primarily applied in: Bone regeneration and repair (including joints and cartilage), Wound healing, and Delaying cellular aging and anti-aging treatments.

There are numerous patents related to these applications, and a detailed discussion is omitted here.

In summary, exploring the mechanisms and therapeutic roles of antler stem cell-derived exosomes in treating and improving various diseases is of great scientific and clinical significance. Further research and development of drugs or therapeutic products based on antler stem cell-derived exosomes hold immense market potential.

Notably, there are currently no patents related to the use of antler stem cell-derived exosomes for improving skin quality and promoting hair growth, highlighting a significant opportunity for innovation in this field.

### SUMMARY OF THE INVENTION

The main purpose of the present invention is to provide a preparation method for exosomes derived from antler periosteal mesenchymal stem cells and their applications, the exosomes produced from these stem cells can enhance skin quality and promote hair growth.

To achieve the aforesaid purpose, the present invention provides a method for preparing exosomes of antler periosteal mesenchymal stem cells, which at least comprises the following steps: Preparation of antlers: Fresh antlers are collected and cleaned. The velvet on the surface of the antlers is scraped off, followed by additional cleaning; Extraction of periosteal tissue: The epidermis of the antler is peeled off to extract the periosteal tissue, which is then placed into a DPBS (Dulbecco's Phosphate-Buffered Saline) culture dish containing antibiotics; Decomposition of periosteal tissue: Residual epidermal tissue and blood on the periosteal tissue are removed. The periosteal tissue is finely chopped and washed with DPBS until no blood remains. The chopped periosteal tissue is then mixed with digestive enzymes and incubated in a CO₂ incubator for decomposition; Isolation of primary periosteal cells: The digested periosteal tissue is added to alpha-MEM complete culture medium, filtered through a cell strainer, and centrifuged. The primary periosteal cells are collected from the cell pellet at the bottom of the centrifuge tube; Cultivation of mesenchymal stem cells: The obtained primary periosteal cells are counted and cultured in a specialized culture medium. The culture medium contains alpha-MEM, 10% fetal bovine serum (FBS), amino acids, basic fibroblast growth factor (bFGF), and gentamicin. The culture medium is replaced every three days, and after seven days, the cells are passaged to obtain antler periosteal mesenchymal stem cells (MSCs), which are then preserved; Cultivation of mesenchymal stem cell-derived exosomes: When the cultured antler periosteal MSCs reach 80% confluency, the cell culture medium is removed, and the cells are washed twice with DPBS. Alpha-MEM is then added, and the cells are cultured continuously for seven days.; Exosome filtration and collection: The alpha-MEM culture medium from the seven-day culture is filtered using a 0.22 μm filter. The exosomes are isolated and concentrated using tangential flow filtration (TFF). The final concentration of exosomes is 1.53×10¹⁰±2.08×10⁹ particles/mL, and the exosomes are stored at −80° C.

The present invention also provides the application of exosomes derived from antler periosteal mesenchymal stem cells in human fibroblasts, promoting fibroblast proliferation and tissue repair.

The present invention further provides the use of exosomes from antler periosteal mesenchymal stem cells in human skin, enhancing skin texture and quality.

The present invention also includes the application of exosomes from antler periosteal mesenchymal stem cells for the formulation of skincare products that improve human skin quality.

The present invention further provides the use of exosomes from antler periosteal mesenchymal stem cells in human hair follicles, promoting hair regeneration and growth.

### BRIEF DESCRIPTION OF THE DRAWINGS

FIG. 1 is a flowchart illustrating the preparation method of the present invention.

FIG. 2 is an analysis diagram of the polymerase chain reaction (PCR) using the marker factors CD9, CD105, Sox2, and CD45 of antler periosteal mesenchymal stem cells in the present invention.

FIGS. 3(a), (b), (c), (d), (e), (f), (g), (h) show immunofluorescence staining performed on antler periosteal mesenchymal stem cells using monoclonal antibodies against marker factors CD29, CD44, CD73, CD90, CD105, CD34, and CD45, with marker factor detection conducted via flow cytometry.

FIG. 4 illustrates the analysis of exosomes derived from antler periosteal mesenchymal stem cells using a Nanoparticle Tracking Analysis (NTA) particle size analyzer.

FIGS. 5(a), (b), (c), (d) present immunofluorescence staining images of exosomes from antler periosteal mesenchymal stem cells, analyzed using monoclonal antibodies targeting exosome-specific markers CD9, CD63, and CD81.

FIGS. 6(a) and (b) show the scratch assay area analysis and corresponding micrographs of co-cultured human fibroblasts treated with exosomes derived from antler periosteal mesenchymal stem cells at different concentrations and time points.

FIG. 7 is a graph showing the variation in skin moisture content at different time points after applying exosomes derived from antler periosteal mesenchymal stem cells.

FIG. 8 is a graph analyzing the changes in sebum secretion at different time points after applying exosomes derived from antler periosteal mesenchymal stem cells to the skin.

FIGS. 9(a) and (b) present photographic evidence of the change in sebum secretion levels before and after four weeks of applying exosomes derived from antler periosteal mesenchymal stem cells to the forehead (the yellow-marked area indicates the sebum distribution).

FIG. 10 is a graph analyzing the variation in skin glossiness at different time points following the application of exosomes derived from antler periosteal mesenchymal stem cells.

FIG. 11 is a graph depicting the change in skin roughness at different time points after applying exosomes derived from antler periosteal mesenchymal stem cells.

FIGS. 12(a) and (b) display photographs of the skin roughness changes before and after four weeks of treatment with exosomes derived from antler periosteal mesenchymal stem cells.

FIG. 13 is a graph illustrating the change in the number of enlarged pores at different time points after applying exosomes derived from antler periosteal mesenchymal stem cells to the skin.

FIGS. 14(a) and (b) show the photographic comparison of enlarged pore reduction before and after four weeks of treatment with exosomes derived from antler periosteal mesenchymal stem cells on facial skin.

FIG. 15 is a graph analyzing changes in skin pigmentation (dark spots) at different time points after applying exosomes derived from antler periosteal mesenchymal stem cells.

FIGS. 16(a) and (b) present photographic evidence of the reduction in dark spots before and after four weeks of applying exosomes derived from antler periosteal mesenchymal stem cells to the skin.

FIG. 17 is a graph showing the variation in fine lines on the skin at different time points after the application of exosomes derived from antler periosteal mesenchymal stem cells.

FIGS. 18(a) and (b) display photographic images showing the reduction of fine lines before and after four weeks of applying exosomes derived from antler periosteal mesenchymal stem cells to facial skin.

FIG. 19 is a graph analyzing changes in the melanin index of the skin at different time points after the application of exosomes derived from antler periosteal mesenchymal stem cells.

FIG. 20 is a graph analyzing the variation in hair density at different time points following the application of exosomes derived from antler periosteal mesenchymal stem cells to the scalp.

FIGS. 21(a), (b), (c), (d) are photographs comparing hair density before and after four weeks of applying exosomes derived from antler periosteal mesenchymal stem cells to the scalp in two test subjects.

FIG. 22 is a graph illustrating the variation in hair shaft diameter at different time points following the application of exosomes derived from antler periosteal mesenchymal stem cells to the scalp.

FIG. 23 is a graph analyzing the change in the melanin index of hair at different time points after the application of exosomes derived from antler periosteal mesenchymal stem cells to the scalp.

FIG. 24 is a graph illustrating the changes in hair growth rate at different time points following the application of exosomes derived from antler periosteal mesenchymal stem cells to the scalp.

### DETAILED DESCRIPTION OF THE INVENTION

Hereinafter, several preferred embodiments of the present invention are cited and described in further detail together with the drawings:

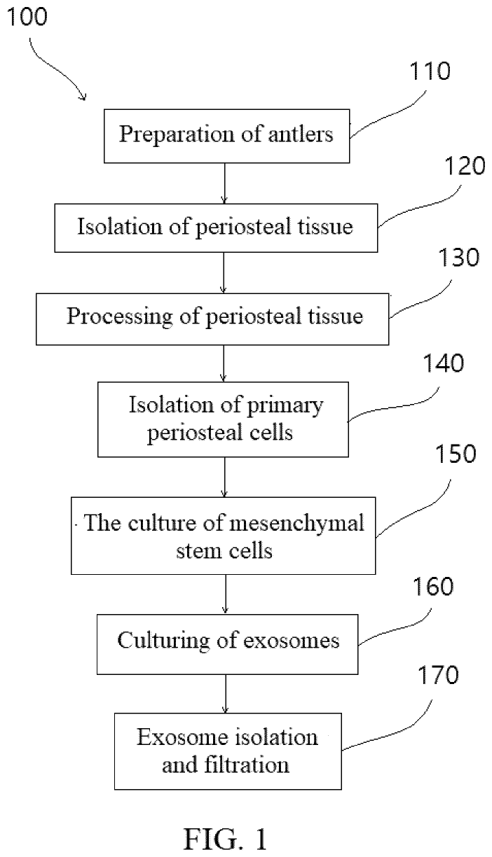

First, please refer to FIG. 1, which illustrates a preferred embodiment (100) of the preparation method for exosomes derived from antler periosteal mesenchymal stem cells in the present invention.

The first step (110) is the preparation of antlers: fresh antlers (1-2 months old) are collected and cleaned using alcohol and sterile gauze to remove stains and residual blood. The antlers are then washed with an antibiotic-containing phosphate-buffered saline (HiMedia Dulbecco's Phosphate Buffered Saline, DPBS). Next, in a Good Distribution Practice (GDP)-compliant laboratory, the velvet is carefully removed from the antler surface using a disposable surgical blade on a sterile workbench, followed by another washing step with antibiotic-containing DPBS.

The second step (120) is the isolation of periosteal tissue: a disposable surgical blade is used to peel off the epidermis of the antler to obtain periosteal tissue, which is then placed in a DPBS culture dish containing antibiotics for temporary storage.

The third step (130) is processing of periosteal tissue: after removing residual epidermal tissue and blood, the periosteal tissue is minced into 1 mm² fragments and washed with DPBS until all blood is removed. Then, 15 ml of digestive enzymes is added for every 3 g of minced periosteal tissue, followed by incubation at 37° C. with 20% carbon dioxide (CO₂) for 30 minutes to facilitate tissue digestion.

The fourth step (140) is the isolation of primary periosteal cells: the digested periosteal tissue is mixed with 20 ml of complete α-MEM culture medium, and a 70 μm cell strainer is used to filter out the isolated periosteal tissue cells. These cells are then centrifuged at 1,500 rpm for 10 minutes to collect the pelleted periosteal primary cells.

The fifth step (150) is the culture of mesenchymal stem cells (MSCs): the periosteal primary cells are counted and cultured using a specially formulated medium, which contains: α-MEM, 10% fetal bovine serum (FBS), 10 mg/ml alanine, 9 mg/ml asparagine, 15 mg/ml aspartic acid, 10 mg/ml glycine, 50 mg/ml glutamic acid, 10 mg/ml proline, 10 mg/ml serine, 10 ng/ml basic fibroblast growth factor (bFGF), and 50 mg/ml gentamicin.

The culture medium is refreshed every three days, and after seven days, the cells are passaged to obtain antler periosteal mesenchymal stem cells (MSCs), which are then stored. Alanine participates in the glucose metabolic pathway, provides energy, and can be converted into other essential organic molecules. Asparagine is involved in protein synthesis, amino acid metabolism, and nitrogen supply. Aspartic acid plays a role in neurotransmission, protein synthesis, and various biochemical processes. Glycine is essential for the synthesis of proteins, collagen, and neurotransmitter precursors. Glutamic acid is involved in amino acid metabolism and excitatory neurotransmission. Proline stabilizes protein structures, particularly collagen. Serine is essential for protein synthesis, biological membrane structure, and metabolic pathways. Basic fibroblast growth factor (bFGF) is a multifunctional growth factor that regulates cell growth, proliferation, and differentiation. In cell culture, it promotes cell proliferation and maintains stem cell self-renewal. Gentamicin is an aminoglycoside antibiotic widely used against Gram-negative bacterial infections, particularly Escherichia coli (E. coli). It functions by binding to the bacterial 30S ribosomal subunit, thereby inhibiting protein synthesis, leading to bacterial growth suppression or cell death.

The sixth step (160) is culturing of exosomes: the antler periosteal mesenchymal stem cells are cultured until they reach 80% confluence, after which the culture medium is removed and the cells are washed twice with DPBS. Fresh α-MEM is added, and the culture is maintained for seven additional days.

The seventh step (170) is the exosome isolation and filtration: the α-MEM medium from the seven-day culture is filtered using a 0.22 μm filter. Exosomes are then isolated and concentrated using tangential flow filtration (TFF). After concentration, the final exosome concentration is measured at 1.53×10¹⁰±2.08×10⁹ particles/ml and stored at −80° C. for long-term preservation.

The exosomes derived from antler periosteal mesenchymal stem cells contain growth factors associated with skin repair, such as epidermal growth factor (EGF), basic fibroblast growth factor (bFGF), and transforming growth factor-beta 1 (TGF-β1). These growth factors were quantified using a Human ELISA kit, and the results indicated the following concentrations in the exosomes of antler periosteal mesenchymal stem cells: EGF: 81.1±14.4 pg/ml, bFGF: 3.4±2.4 pg/ml and TGF-β1: 1236.0±67.0 pg/ml.

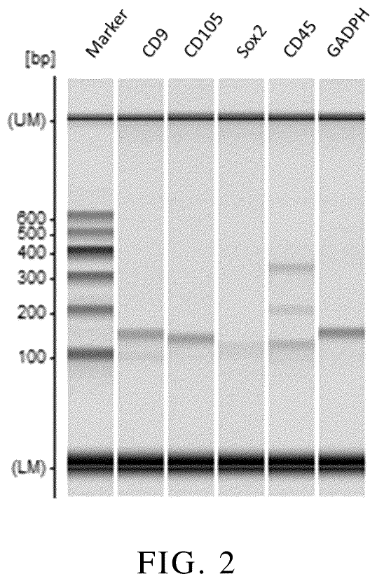

In addition, the present invention provides a species identification method for antler periosteal mesenchymal stem cells. The mesenchymal stem cells obtained from the cell culture process (Step 150) were analyzed using polymerase chain reaction (PCR) with antler mesenchymal stem cell-specific markers, including CD9, CD105, Sox2, and CD45, as shown in FIG. 2.

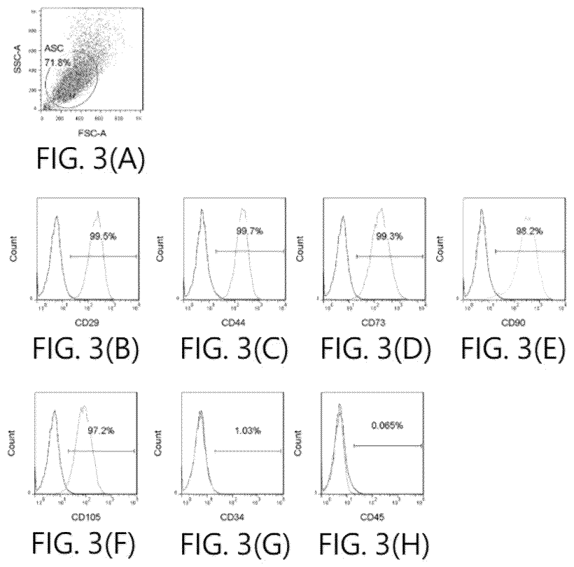

Furthermore, immunofluorescence staining was performed on antler periosteal mesenchymal stem cells using monoclonal antibodies targeting the surface markers CD29, CD44, CD73, CD90, CD105, CD34, and CD45. The expression levels of these markers were quantified using flow cytometry (FACS analysis), as illustrated in FIG. 3.

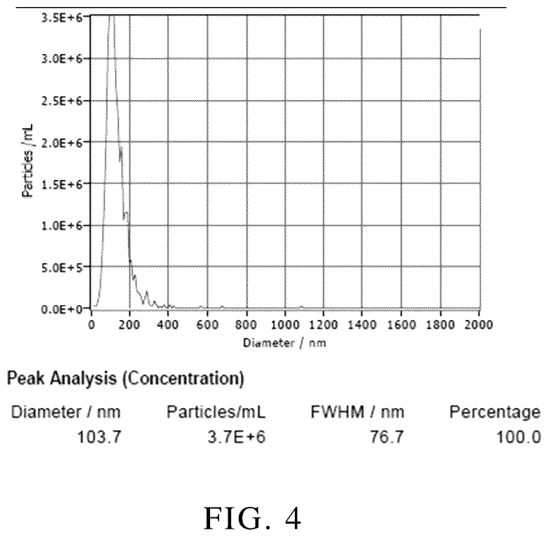

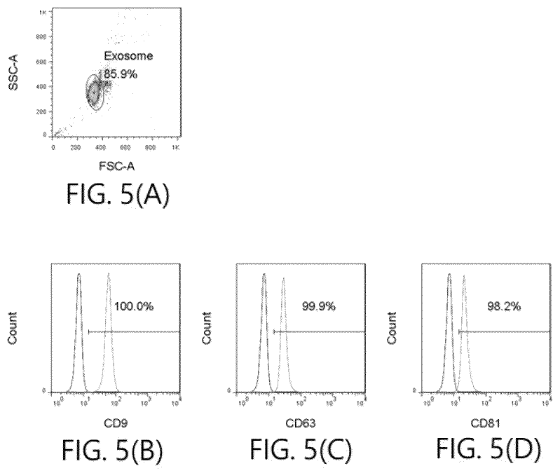

Moreover, the present invention provides a method for identifying exosomes derived from antler periosteal mesenchymal stem cells. The size distribution of the exosomes was analyzed using a nanoparticle tracking analysis (NTA) system, as shown in FIG. 4. The peak particle diameter of the exosomes was 103.7 nm. Additionally, immunofluorescence staining was conducted using exosome-specific monoclonal antibodies against CD9, CD63, and CD81, as demonstrated in FIG. 5.

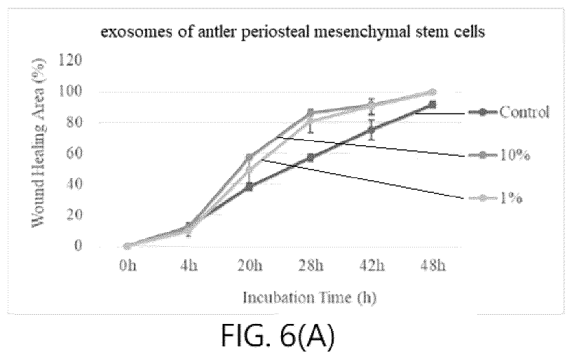

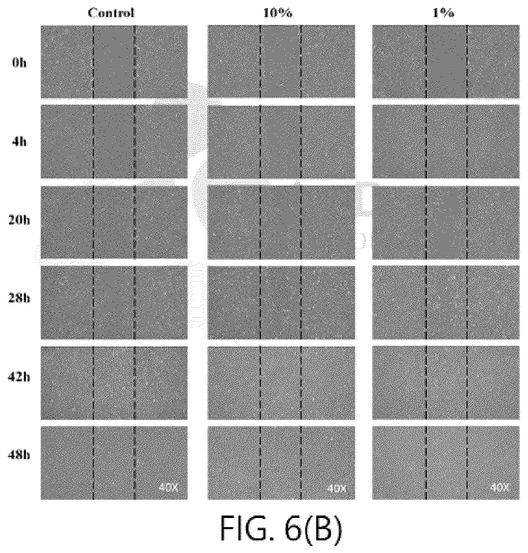

In another embodiment, the present invention provides an application of exosomes derived from antler periosteal mesenchymal stem cells in promoting human fibroblast proliferation and repair. Human fibroblasts were incubated with exosomes derived from antler periosteal mesenchymal stem cells at a concentration range of 1%-10% for 20 hours. The results demonstrated that the exosomes significantly enhanced fibroblast migration toward the scratch area. After 48 hours, the scratch area in the exosome-treated group was almost completely repopulated with fibroblasts, as shown in FIG. 6, suggesting the potential of these exosomes in treating skin defects and related conditions.

In yet another embodiment, the present invention describes an application of exosomes derived from antler periosteal mesenchymal stem cells for enhancing human skin texture.

In certain embodiments, the present invention also proposes the use of exosomes derived from antler periosteal mesenchymal stem cells in skincare and cosmetic formulations to improve skin quality.

In another embodiment, the present invention provides the application of exosomes derived from antler periosteal mesenchymal stem cells to stimulate hair growth.

In some embodiments, the present invention further provides the use of exosomes derived from antler periosteal mesenchymal stem cells in the formulation of hair growth-promoting products.

Description of Experiments on Skin and Hair Growth Using Exosomes from Antler Periosteal Mesenchymal Stem Cells

Skin Test:

Subjects:

Volunteers were recruited as test subjects. A total of 30 individuals participated, including 25 women and 5 men, with ages ranging from 22 to 55 years old and an average age of 32.4 years.

Test Conditions: Skin measurements were conducted under a relative humidity of 55±5% and a room temperature of 25±1° C.

Efficacy Testing Methods:

1. Test subjects washed their faces and waited for the moisture to dry naturally before undergoing various skin assessments.

2. A Visia Complexion Analysis system (Canfield Scientific, Inc., USA) was used to measure skin spots, wrinkles, fine lines, roughness, and enlarged pores.

3. Skin elasticity was assessed using the Aramo TS Integrated Skin Diagnosis System (Aram HUVIS Co., Ltd., Korea).

4. Skin brightness (L-value) was measured using a Minolta Chromameter (CM2500d, Japan).

5. Skin moisture content was measured using a C+K Corneometer CM 825 (Courage-Khazaka Electronic, Germany).

6. Skin glossiness was measured using a C+K Glossymeter GL 200 (Courage-Khazaka Electronic, Germany).

7. Skin melanin index was measured using a Derma-Spectrophotometer (Cortex Technology, Hadsund, Denmark).

8. Exosomes from antler periosteal mesenchymal stem cells were applied to the skin every morning and evening. Skin texture changes were assessed at scheduled intervals using the aforementioned measurement instruments.

9. Changes in various skin parameters before and after applying the antler periosteal mesenchymal stem cell exosomes were compared.

Evaluation Results (Facial Efficacy):

Before use (W0), baseline skin texture was examined. Subjects applied antler periosteal mesenchymal stem cell exosomes to the entire face every morning and evening. Skin texture changes were assessed weekly at W1 (Week 1), W2 (Week 2), W3 (Week 3), and W4 (Week 4) using a skin analysis system.

The measured parameters included moisture content, sebum secretion, glossiness, roughness, number of pores, skin spots, fine lines, melanin index, and other indicators of skin condition.

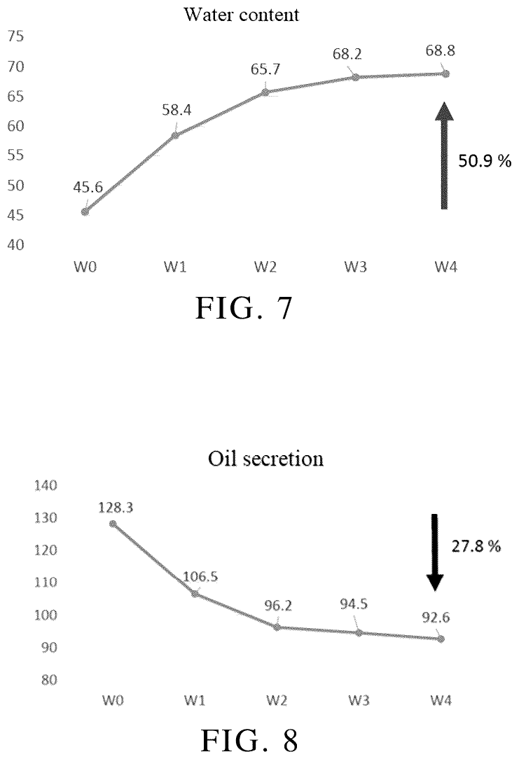

Moisture Content: As shown in FIG. 7, the average moisture content was: Before use (W0): 45.6±5.8, week 1 (W1): 58.4±8.6, week 2 (W2): 65.7±10.2, week 3 (W3): 68.2±6.7 and week 4 (W4): 68.8±8.8. After 4 weeks, the total average skin moisture content increased by 23.2%, with an overall improvement of 50.9% compared to baseline.

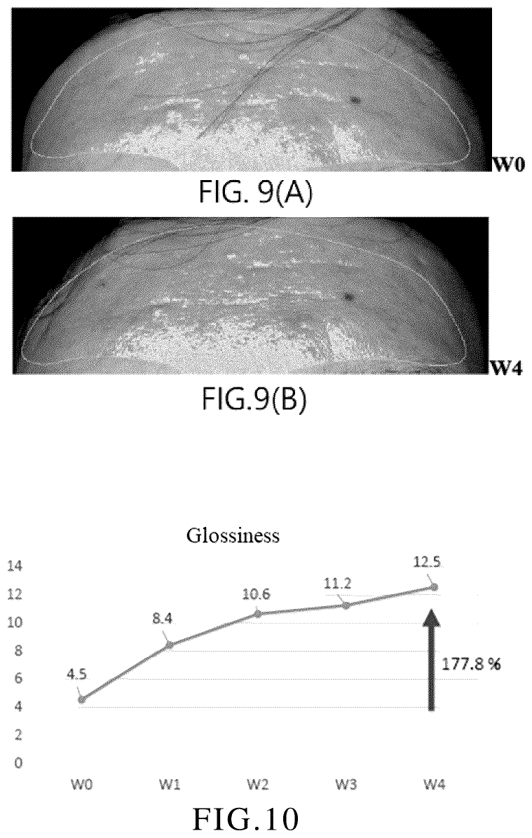

Sebum Secretion: As shown in FIG. 8, the average sebum secretion level was: Before use (W0): 128.3±9.8, week 1 (W1): 106.5±12.5, week 2 (W2): 96.2±8.7, week 3 (W3): 94.5±9.5 and week 4 (W4): 92.6±11.2. Over the 4-week period, the total average sebum secretion decreased by 35.7 units, achieving a 27.8% reduction in sebum production. FIG. 9 shows a test case illustrating the reduction in forehead oiliness. The yellow-highlighted areas indicate the initial oil distribution before application and the improvement after 4 weeks of continuous use.

Glossiness: As shown in FIG. 10, the skin glossiness index was: Before use (W0): 4.5±2.6, week 1 (W1): 8.4±1.4, week 2 (W2): 10.6±1.6, week 3 (W3): 11.2±2.5 and week 4 (W4): 12.5±2.8. Over 4 weeks, the total average glossiness increased by 8.0%, achieving an improvement of 177.8%.

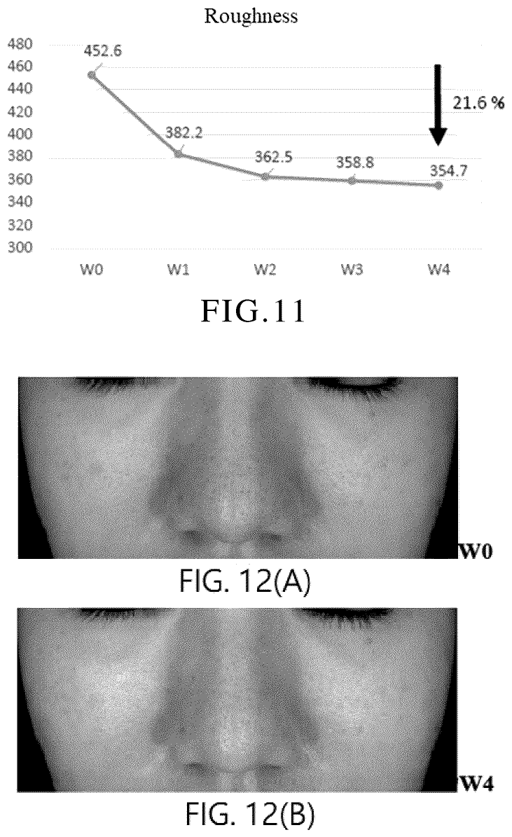

Skin Roughness: As shown in FIG. 11, the skin roughness index was: Before use (W0): 452.6±31.8, week 1 (W1): 382.2±45.2, week 2 (W2): 362.5±36.8, week 3 (W3): 358.8±32.7 and week 4 (W4): 354.7±28.8. After 4 weeks, the total average roughness decreased by 97.9, showing a 21.6% improvement in skin texture. FIG. 12 shows a test case demonstrating the improvement in skin roughness. Before use, pores on the cheeks and nose appeared rough and uneven. After 4 weeks of continuous use, the skin became noticeably smoother and brighter.

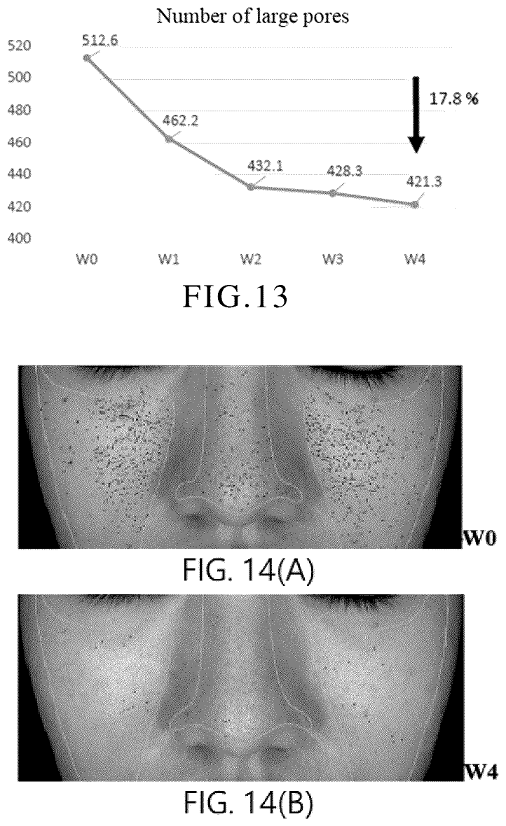

Number of Enlarged Pores: As shown in FIG. 13, the number of enlarged pores was: Before use (W0): 512.6±58.6, week 1 (W1): 462.2±52.5, week 2 (W2): 432.1±48.9, week 3 (W3): 428.3±55.2 and week 4 (W4): 421.3±46.6. After 4 weeks, the total number of enlarged pores decreased by 91.3, achieving a 17.8% reduction. FIG. 14 shows a test case illustrating the reduction in pore size and number after 4 weeks of continuous use.

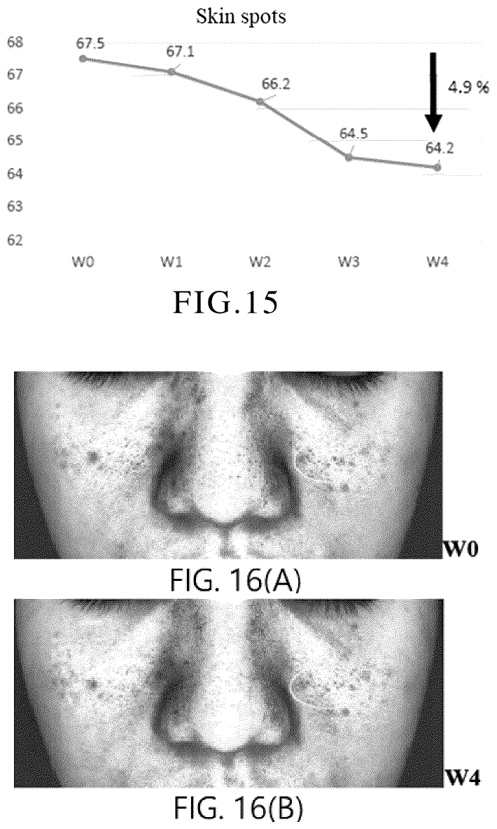

Skin Spots: As shown in FIG. 15, the skin spot index was: Before use (W0): 67.5±22.4, week 1 (W1): 67.1±20.6, week 2 (W2): 66.2±18.8, week 3 (W3): 64.5±16.7 and week 4 (W4): 64.2±15.4. After 4 weeks, the total average number of skin spots decreased by 3.3%, achieving a 4.9% improvement. FIG. 16 shows a test case where skin spots slightly faded after 4 weeks of continuous use.

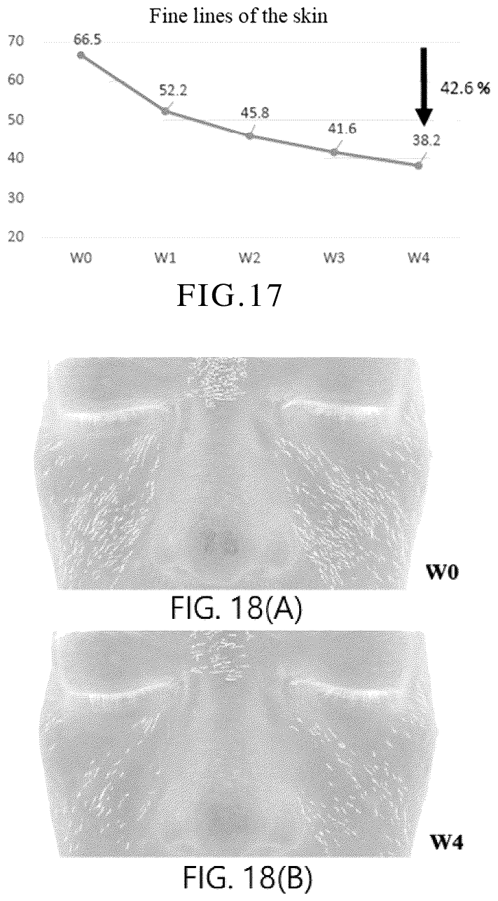

Fine Lines: As shown in FIG. 17, the fine lines index was: Before use (W0): 66.5±11.5, week 1 (W1): 52.2±12.4, week 2 (W2): 45.8±12.7, week 3 (W3): 41.6±10.5 and week 4 (W4): 38.2±10.2. After 4 weeks, the total average fine lines decreased by 28.3%, achieving a 42.6% improvement. FIG. 18 illustrates a reduction in fine lines after 4 weeks of continuous use.

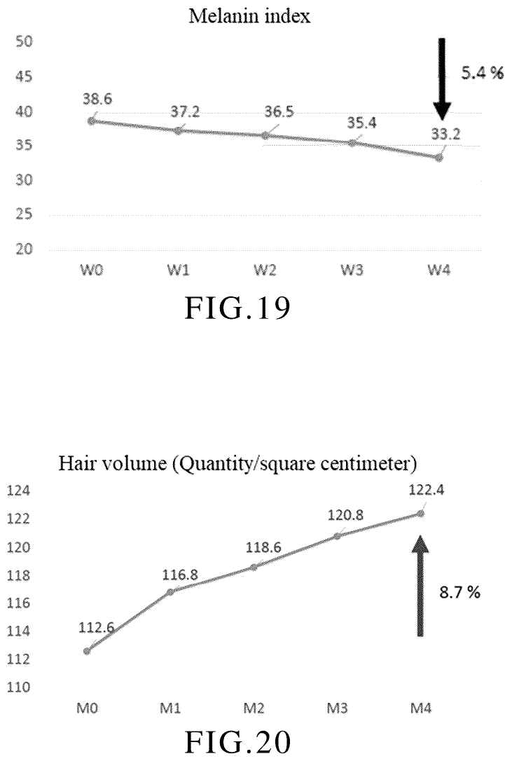

Melanin Index: As shown in FIG. 19, the melanin index slightly decreased by 14.0 units, resulting in a 5.4% improvement over 4 weeks.

Hair Growth Test:

Subjects:

Volunteers were recruited as test subjects. A total of 30 participants were tested, including 25 females and 5 males, aged between 22 and 55 years, with an average age of 32.4 years.

Test Conditions:

Hair and scalp measurements were conducted under a relative humidity of 55±5% and a room temperature of 25±1° C.

Efficacy Testing Methods:

1. Baseline measurements of hair and scalp conditions were taken before the test.

2. Hair volume, hair diameter, and other hair/scalp-related parameters were measured using the Hair/Scalp Consultation System (Aramo TS, Hair Diagnosis System, Aram HUVIS Co., Ltd., Korea).

3. Exosomes derived from antler periosteal mesenchymal stem cells were applied daily for a period of 4 to 5 months.

4. Changes in hair and scalp parameters before and after the application of exosomes were compared.

Evaluation Results (Hair Growth Efficacy):

Before applying the test sample, hair volume was measured and recorded using the hair diagnostic system. Subsequent measurements were conducted 1 month, 2 months, 3 months, and 4 months (or 5 months) after application using the same method. Results were expressed as the number of hairs per square centimeter (cm²). The evaluated parameters included hair volume, follicle detection, hair diameter, melanin index, growth rate, and other hair-related indicators.

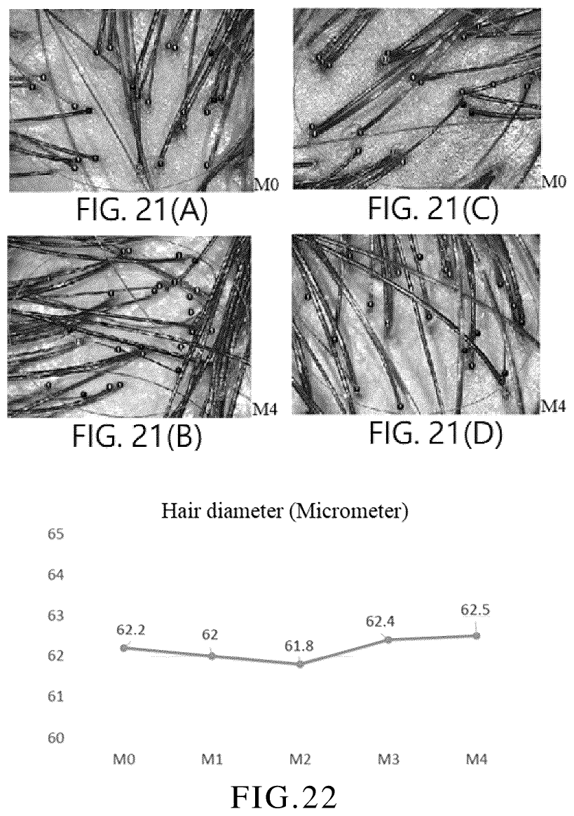

Hair Volume: As shown in FIG. 20, the average hair volume was: Before use (M0): 112.6±25.7, after 1 month (M1): 116.8±27.2, after 2 months (M2): 118.6±27.5, after 3 months (M3): 120.8±24.1 and after 4 months (M4): 122.4±18.5. Over the 4-month period, the total average hair volume increased by 9.8%, showing an efficacy improvement of 8.7%. As shown in FIG. 21, test cases demonstrated an increasing trend in hair volume after 4 weeks of continuous use.

Hair Diameter: As shown in FIG. 22, the average hair diameter was: Before use (M0): 62.2±2.5 μm, after 1 month (M1): 62.0±1.7 μm, after 2 months (M2): 61.8±1.2 μm, after 3 months (M3): 62.4±1.5 μm and after 4 months (M4): 62.5±1.1 μm, During the 4-month period, hair diameter did not show significant changes.

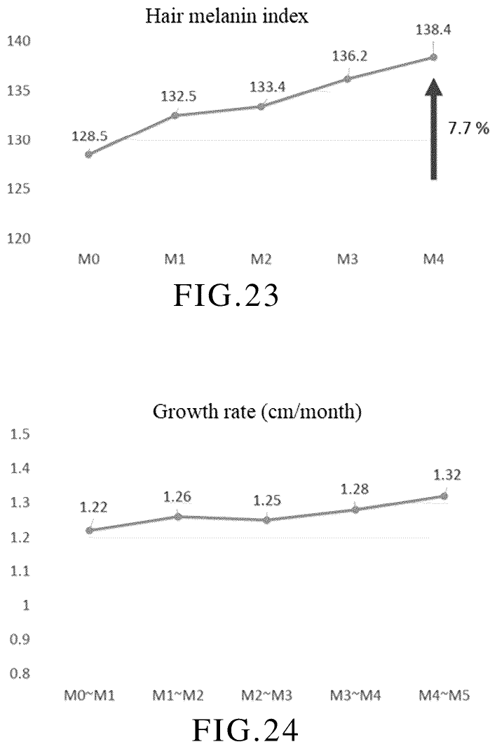

Hair Melanin Index: As shown in FIG. 23, the average melanin index was: Before use (M0): 128.5±21.6, after 1 month (M1): 132.5±15.8, after 2 months (M2): 133.4±18.5, after 3 months (M3): 136.2±25.1 and after 4 months (M4): 138.4±27.2. During the 4-month period, the melanin index of hair slightly increased by 7.7%, indicating a gradual enhancement of hair pigmentation.

Hair Growth Rate: As shown in FIG. 24, the average hair growth rate was: After 1 month (M0˜M1): 1.22±0.02 cm, after 2 months (M1˜M2): 1.26±0.03 cm, after 3 months (M2˜M3): 1.25±0.03 cm, after 4 months (M3˜M4): 1.28±0.02 cm and after 5 months (M4˜M5): 1.32±0.02 cm. Throughout the test period, the hair growth rate showed a slight increase over time.

Based on the above findings, the exosomes derived from antler periosteal mesenchymal stem cells exhibited the following effects:

For skin improvement: Significant increases in skin moisture content and glossiness. Substantial reductions in sebum secretion, skin roughness, number of enlarged pores, and fine lines. Improvements in skin spots and melanin index.

For hair growth enhancement: Increase in hair volume. Enhancement in hair pigmentation (melanin index). Improvement in hair growth rate.

These results confirm that the exosomes from antler periosteal mesenchymal stem cells effectively improve skin texture and promote hair growth.

The examples mentioned above are merely some of the preferred embodiments of this invention and are not intended to limit its scope. Any person skilled in the relevant technical field can make modifications, alterations, and embellishments without departing from the spirit and scope of this invention. Therefore, the scope of protection of this invention should be defined by the appended patent claims.

What is claimed is:

1. A method for preparing exosomes from antler periosteal mesenchymal stem cells, comprising at least the following steps:
preparation of antlers: harvest fresh antlers that have grown for 1 to 2 months and wash them with DPBS (Dulbecco's Phosphate-Buffered Saline) containing antibiotics, then, completely remove the velvet from the surface of the antlers and wash them again with DPBS containing antibiotics;
isolation of periosteal tissue: peel off the outer skin of the antlers to extract the periosteal tissue, place the periosteal tissue into a DPBS culture dish containing antibiotics for further processing;
processing of periosteal tissue: remove any remaining epidermal tissue and blood from the periosteal tissue, mince the periosteal tissue into fragments of approximately 1 mm² in size, and wash the minced tissue with DPBS until no visible blood remains, then add 15 ml of digestive enzymes per 3 g of minced periosteal tissue and incubate at 37° C. in a 20% CO₂ incubator for 30 minutes to facilitate enzymatic digestion of the periosteal tissue;
isolation of primary periosteal cells: transfer the digested periosteal tissue into 20 ml of alpha-MEM complete culture medium, filter the cell suspension through a 70 μm cell strainer to separate periosteal cells, and centrifuge the filtrate at 1500 rpm for 10 minutes to collect the primary periosteal cells as a cell pellet;
culture of mesenchymal stem cells: count the isolated periosteal primary cells and culture them in a medium containing alpha-MEM, 10% fetal bovine serum (FBS), 10 mg/ml alanine, 9 mg/ml asparagine, 15 mg/ml aspartic acid, 10 mg/ml glycine, 50 mg/ml glutamic acid, 10 mg/ml proline, 10 mg/ml serine, 10 ng/ml basic fibroblast growth factor-2 (bFGF-2), and 50 mg/ml gentamicin, then replace the culture medium every three days, after seven days, passage the cells to expand the population and obtain mesenchymal stem cells (MSCs) derived from antler periosteum, which are then preserved;
culturing of exosomes: when the antler periosteal MSCs reach 80% confluence, remove the culture medium, wash the cells twice with DPBS, and replace it with fresh alpha-MEM, continue culturing for 7 days; and
exosome isolation and filtration: after 7 days of culture, filter the alpha-MEM supernatant through a 0.22 μm filter, the exosomes are then isolated and concentrated using tangential flow filtration (TFF) with a selective membrane, the final exosome concentration is 1.53×10¹⁰±2.08×10⁹ particles/ml, the purified exosomes are stored at −80° C. for preservation.

2. An exosome Derived from Antler Periosteal Mesenchymal Stem Cells which are prepared using the method as described in claim 1, the obtained exosomes have a particle size ranging from 50 to 400 nm and contain the following growth factors: EGF content: 81.1±14.4 pg/ml, bFGF content: 3.4±2.4 pg/ml and TGF-β1 content: 1236.0±67.0 pg/ml.

3. The exosomes derived from antler periosteal mesenchymal stem cells as described in claim 2, wherein the exosomes were analyzed using a nanoparticle tracking analysis (NTA) system, and the peak particle size was measured at 103.7 nm.

4. The use of exosomes derived from antler periosteal mesenchymal stem cells as described in claim 2 are applied to human fibroblasts to promote their proliferation and facilitate tissue repair.

5. The use of exosomes derived from antler periosteal mesenchymal stem cells as described in claim 3 are applied to human fibroblasts to promote their proliferation and facilitate tissue repair.

6. The use of exosomes derived from antler periosteal mesenchymal stem cells as described in claim 2 are applied to human skin to enhance skin quality. The efficacy indicators for skin quality improvement include skin hydration, sebum secretion, skin radiance, roughness, pore count, pigmentation spots, fine lines, and melanin content.

7. The use of exosomes derived from antler periosteal mesenchymal stem cells as described in claim 3 are applied to human skin to enhance skin quality. The efficacy indicators for skin quality improvement include skin hydration, sebum secretion, skin radiance, roughness, pore count, pigmentation spots, fine lines, and melanin content.

8. The use of exosomes derived from antler periosteal mesenchymal stem cells as described in claim 2 are applied to human hair to promote hair growth. The efficacy indicators for hair growth promotion include hair density, hair follicle assessment, hair shaft diameter, melanin index, and hair growth rate.

9. The use of exosomes derived from antler periosteal mesenchymal stem cells as described in claim 3 are applied to human hair to promote hair growth. The efficacy indicators for hair growth promotion include hair density, hair follicle assessment, hair shaft diameter, melanin index, and hair growth rate.
